(19) United States
(12) Patent Application Publication
(10) Pub. No.: US 2011/0319749 A1
(43) Pub. Date: Dec. 29, 2011

(54) System and Method for Determining Medical Capsule Location inside a Human Body

(75) Inventors: Kang-Huai Wang, Saratoga, CA (US); Jiafu Luo, Irvine, CA (US); Gordon C. Wilson, San Francisco, CA (US)

(73) Assignee: CAPSO VISION, INC., Saratoga, CA (US)

(21) Appl. No.: 12/822,074

(22) Filed: Jun. 23, 2010

(51) Int. Cl. A61B 5/05

(52) U.S. Cl. 600/424

(57) ABSTRACT

Systems and methods are provided for capsule location determination. There is a need to estimate the capsule location in a living body for drug dispensing, correlating capsule images with actual tract location and other purposes. A capsule location determining system is disclosed which comprises a plurality of sensing devices and a base station. Each sensing device comprises a radiating element to radiate an inducing signal, a generating circuit to provide a patterned signal to drive the radiating element, a sensing element to sense the induced field and a receiving circuit to provide a recovered signal. The base station estimates the capsule location based on a time profile of collected information related to the recovered signals from all sensing devices. Methods associated with the capsule location determining systems are also disclosed.

### CROSS REFERENCE TO RELATED APPLICATIONS

The present invention is related to US patent application, entitled “Detection of When a Capsule Camera Enters into or Goes out of a Human Body and Associated Operations”, Ser. No. 11/625,647, filed on Jan. 22, 2007, and US patent application, entitled “Multi-Stream Image Decoding Apparatus and Method”, Ser. No. 12/127,753, filed on May 27, 2008. The U.S. Non-Provisional patent applications are hereby incorporated by reference in their entireties.

### FIELD OF THE INVENTION

The present invention relates to medical capsule inside a human body. In particular, the present invention relates to determining the capsule location inside the human body using sensing devices.

### BACKGROUND

Capsule cameras have been widely used in the medical field as an alternative to the conventional endoscopy to examine the gastrointestinal tract. The camera is housed in a swallowable capsule, along with a radio transmitter for transmitting data, primarily comprising images recorded by the digital camera, to an external receiver/data recorder or an on-board storage to store the captured images. The capsule may also include a radio receiver for receiving instructions or other data from a base-station transmitter. The capsule travels through the gastrointestinal tract and will be evacuated from the human body after long hours. The speed of the capsule movement inside the body varies greatly from person to person. Also the speed varies greatly for different parts of the GI tract. For example, the average transit time for a capsule camera to travel through the small bowel is about 5 hours while the average transit time to travel through the colon is about 30 hours. Even in the same section of the tract, a capsule may move forward in one moment and hesitate in the same location for a while in the next moment. Therefore, it is unlikely to determine the capsule location simply based on the time period it traveled.

In many capsule applications, there is a need to know the location of the capsule inside the body. For example, a capsule camera may be used to image the small intestine or the colon. The images captured by the capsule camera are used by a diagnostician to assess any possible anomaly in the tract being imaged. The images captured have to be correlated to the corresponding location of the tract. Therefore, the location of the capsule camera when an associated image is taken is important. It is very desirable to obtain the location information along the course of imaging through an intended section of the tract, such as the small intestine or the colon.

Besides the capsule camera, there are also other types of medical capsules that are ingested into a human body for monitoring or examining purposes. For example, a medical capsule can be used to monitor the pH value of the gastrointestinal tract or temperature of different human organs. A medical capsule may also be used to deliver medicines to desired spots inside the human body or collect samples, such as body fluids, inside a human body. Therefore, it is desirable to know the location of the medical capsule inside the human body so that the capsule can monitor/measure data, dispense medicine, or collect samples at the intended location of the tract.

After a capsule is ingested into the human body, it will eventually be evacuated from the human body. Nevertheless, in rare cases, the capsule may be stuck in the tract and requires medical attention. If such condition is not treated timely and properly, it may cause dangerous side effects. Some capsule types need to be retrieved after the capsules exist from the human body. For example, a capsule camera with on-board storage has to be retrieved to read out the images stored on-board. It is desirable that it can be determined when the capsule camera is near the rectum/anus, and therefore, the patient can be prepared with a procedure for retrieving the capsule.

Various methods to detect the location of a medical capsule have been described in prior arts. The US patent application, entitled “Detection of When a Capsule Camera Enters into or Goes out of a Human Body and Associated Operations”, Ser. No. 11/625,647, filed on Jan. 22, 2007 discloses a capsule that can detect the moment of capsule exiting from the body and provides alert. The US patent application, entitled “Multi-Stream Image Decoding Apparatus and Method”, Ser. No. 12/127,753, filed on May 27, 2008 discloses a multi-stream image recoding system where multiple recorders are used to receive capsule data through a wireless link The network condition measured by Receiver Signal Strength Indication (RSSI) is used to manage the recording of multiple streams. The network condition assessment also indirectly provides location indication based on RSSI, i.e., the network assessment identifies the recorders that are likely the closest to the capsule inside the body.

In U.S. Pat. No. 7,144,366, entitled “Capsule Medical Apparatus Having Evacuation Detecting and Notifying Devices and Capsule Medical Apparatus Collecting System” by Takizawa et al., several methods are described for detecting when a medical capsule is being evacuated from the human body or is about to be evacuated. One method disclosed utilizes an extracorporeal unit having an antenna to transmit an electric wave to the medical capsule. The medical capsule detects the strength of the electric wave and provides a notification signal to the extracorporeal unit. Another method disclosed places a magnet inside the medical capsule. The extracorporeal unit has a sensor to detect the strength of the magnetic field from the capsule inside the body and to provide a notification signal based on the strength of the magnetic field. The apparatus developed by Takizawa et al., only provides rudimentary location information regarding when a medical capsule is being or is about to be evacuated from the human body. It does not provide accurate location information. Furthermore, the electric wave from the extracorporeal unit or the magnetic field from the magnet inside the medical capsule is susceptible to interfering sources in the surrounding environment. The US patent application, entitled “System and Method for Controlling a Device in vivo”, Ser. No. 10/252,826, filed on Sep. 24, 2002 by Lewkowicz at el., discloses a capsule location determining and steering system utilizing a steerable transceiver. The steerable transceiver can transmit a positional signal and receive a signal having a vector. The location of the capsule can be determined with reasonable accuracy. Nevertheless, the steerable transceiver is quite large and requires a patient to be in a special facility for determining the capsule location, which is very inconvenient.

In light of the above discussion, it is apparent that there is a need for a light-weight and accurate location determining system for a medical capsule inside a human body.

### BRIEF SUMMARY OF THE INVENTION

The present invention discloses a system for determining the location of a medical capsule ingested into a living body. The medical capsule location determining system comprises a medical capsule adapted to be ingested into a living body, a plurality of sensing devices, and a base station. Each of the sensing devices comprises a radiating element to radiate an inducing field into the living body, a signal generating circuit to provide a patterned signal to drive the radiating element, a sensing element to sense an induced field caused by the medical capsule inside the living body, and a signal receiving circuit coupled to the sensing element to receive the induced field and to provide a recovered signal associated with the induced field. The base station comprises a processing module to estimate capsule location based on a time profile of collected information, wherein the collected information comprises information related to the recovered signals by the plurality of sensing devices.

While the collected information comprises information related to the recovered signal, the collected information may further comprise information related to the patterned signal. Furthermore, each of the plurality of sensing devices further comprises a controller to evaluate proximity data to include in the collected information. In one embodiment of the present invention, the base station further comprises a notification module to provide notification information based on the capsule location. Each of the plurality of sensing devices further comprises a sensor-side transmitter and the base station further comprises a base-side receiver, wherein the sensor-side transmitter and the base-side receiver communicate through a wired link or a wireless link. Various patterned signals are disclosed to drive the radiating element including a very low frequency (VLF) electrical signal, a pulse induction (PI) signal, and a beat-frequency oscillator (BFO) signal. The inducing field can be an electromagnetic field. The radiating element and the sensing element can be co-located to allow integration into a combined sensing head.

In yet another embodiment according to the present invention, a communication link is provided from the base station to the sensing devices. This communication link allows the base station to provide system management for the plurality of sensing devices. The communication link can be either wireless or wired according to communication link from the plurality of sensing devices to the base station. In still another embodiment according to the present invention, the base station provides capsule management information to the medical capsule wherein the capsule management information may include a time stamp, a command or capsule location.

The present invention discloses another system for determining the location of a medical capsule ingested into a living body. The medical capsule system comprises a medical capsule adapted to be ingested into a living body, and a plurality of sensing devices. Each sensing device comprises a radiating element to radiate an inducing field into the living body, a signal generating circuit to provide a patterned signal to drive the radiating element, a sensing element to sense an induced field caused by the medical capsule inside the living body, a signal receiving circuit coupled to the sensing element to receive the induced field and to provide a recovered signal associated with the induced field, a processor and a transceiver module. A communication network is formed using the transceiver modules to communicate device information among the plurality of sensing devices. Furthermore, the processors are used to estimate capsule location based on a time profile of the device information related to the recovered signals by the plurality of sensing devices.

The present invention also discloses a method for determining the location of a medical capsule ingested into a living body. The method comprises providing a medical capsule, wherein the medical capsule is adapted to be ingested into a living body, placing a plurality of sensing devices over the living body, and estimating capsule location based on a time profile of collected information. Each of the sensing devices radiates an inducing field into the living body according to a patterned signal, senses an induced field caused by the medical capsule inside the living body, and converts the induced field into a recovered signal associated with the induced field. The collected information comprises information related to the recovered signals by the plurality of sensing devices.

The present invention also discloses a alternative method for determining the location of a medical capsule ingested into a living body. The method comprising providing a medical capsule, wherein the medical capsule is adapted to be ingested into a living body, placing a plurality of sensing devices over the living body, providing a communication network using the transceiver modules to communicate device information among the plurality of sensing devices, and estimating capsule location using the processors based on a time profile of the device information. Each of the sensing devices radiates an inducing field into the living body according to a patterned signal, senses an induced field caused by the medical capsule inside the living body, converts the induced field into a recovered signal associated with the induced field, provides a processor, and provides a transceiver module. The time profile of the device information comprises information related to the recovered signals by the plurality of sensing devices.

### BRIEF DESCRIPTION OF THE DRAWINGS

FIG. 1 illustrates an environment of a medical capsule inside a human body where the location of the medical capsule is to be determined.

FIG. 2A illustrates an exemplary sensing device which can be used to evaluate proximity data of a medical capsule inside a living body according to the present invention.

FIG. 2B illustrates a medical capsule location determining system according to one embodiment of the present invention.

FIG. 3A illustrates an exemplary block diagram of a capsule location determining system having a plurality of sensing devices and a base station.

FIG. 3B illustrates an exemplary block diagram of a capsule location determining system having a plurality of sensing devices and a base station, wherein the sensing devices and the base station are connected through a wired link

FIG. 3C illustrates an exemplary block diagram of a capsule location determining system having a plurality of sensing devices and a base, wherein the sensing devices and the base station are connected through a wireless link

FIG. 3D illustrates an exemplary block diagram of a capsule location determining system, similar to FIG. 3C, having a plurality of sensing devices and a base station, wherein the base station are connected further comprises a notification module.

FIG. 4A illustrates an exemplary block diagram of a sensing device including a controller to evaluate proximity data based on information related to the patterned signal and the recovered signal.

FIG. 4B illustrates an exemplary block diagram of a sensing device including a controller to evaluate proximity data based on information related to the recovered signal.

FIG. 5 illustrates another medical capsule location determining system according to one embodiment of the present invention, wherein the sensing devices work collaboratively to determine the medical capsule location.

FIG. 6A illustrates an exemplary block diagram of a medical capsule location system using a plurality of sensing device, wherein the sensor devices are connected using a wired link

FIG. 6B illustrates an exemplary block diagram of a medical capsule location system using a plurality of sensing device, wherein the sensor devices are connected using a wireless link

FIG. 6C illustrates an exemplary block diagram of a medical capsule location system using a plurality of sensing device, wherein the sensor devices are connected using a wireless link and each sensing device comprises a notification module.

FIG. 7 is a flow chart illustrating an exemplary method of medical capsule location determination using a plurality of sensing devices and a base station.

FIG. 8 is a flow chart illustrating an exemplary method of medical capsule location determination using a plurality of sensing devices.

### DETAILED DESCRIPTION OF THE INVENTION

In the disclosure, various embodiments and examples of the methods and structures mentioned above are described. It will be realized that this detailed description is illustrative only and is not intended to be in any way limiting. Other embodiments will readily suggest themselves to persons of ordinary skill in the art having the benefit of this disclosure.

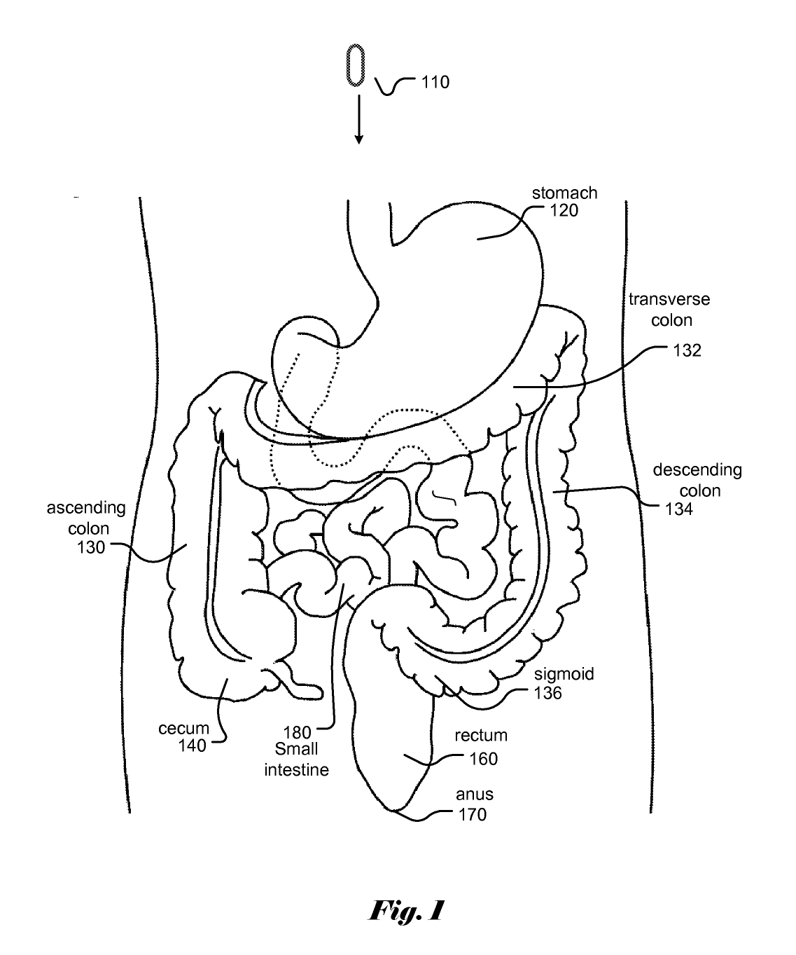

FIG. 1 illustrates an environment that a medical capsule will encounter after it is ingested into the human body. The medical capsule will travel through various parts of the gastrointestinal tract, including esophagus (not shown), stomach 120, small intestine 180, cecum 140, ascending colon 130, transverse colon 132, descending colon 134, sigmoid 136, and rectum 160. The medical capsule will exit from the living body through anus 170 in a normal case. In case that a capsule does not exit from the body within an expected time period, there may be a need to know where the capsule is stuck in the living body. In another scenario, it may be of interest to know when the capsule is about to exit from the body so that preparation can be made to retrieve the capsule. For example, if the medical capsule is a capsule camera having on-board storage, it has to be retrieved to recover the on-board images. When a capsule camera is used as the medical capsule, it is desirable to know the location of the capsule camera so that the pictures being captured can be correlated to the respective parts of the tract. Furthermore, the operation mode of a capsule camera may be controlled by a base station since the base station may evaluate the location of the capsule and provide needed command or information to the capsule camera accordingly. For example, if the capsule camera is intended for imaging the colon, the camera can be initially programmed to a sleep mode after admission wherein the capsule camera may be in a very low power mode with a receiver stayed on to receive command from the base station. Consequently it will conserve the battery power of the capsule camera which is a precious resource for such applications. When the base station determines that the capsule camera enters the colon, the base station transmits a command to wake up the capsule camera to enter a capture mode. When a drug dispensing capsule is used, it is desirable to know the location of capsule so that the drug can be correctly released in the intended location of the tract.

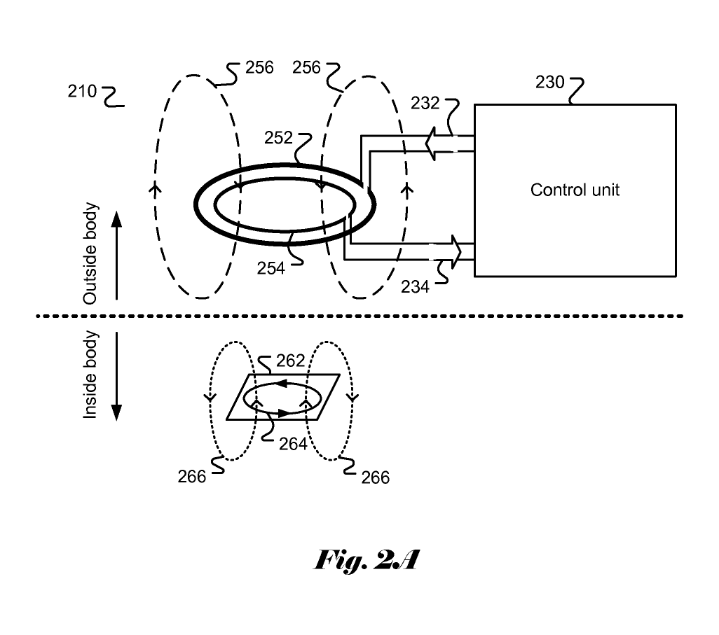

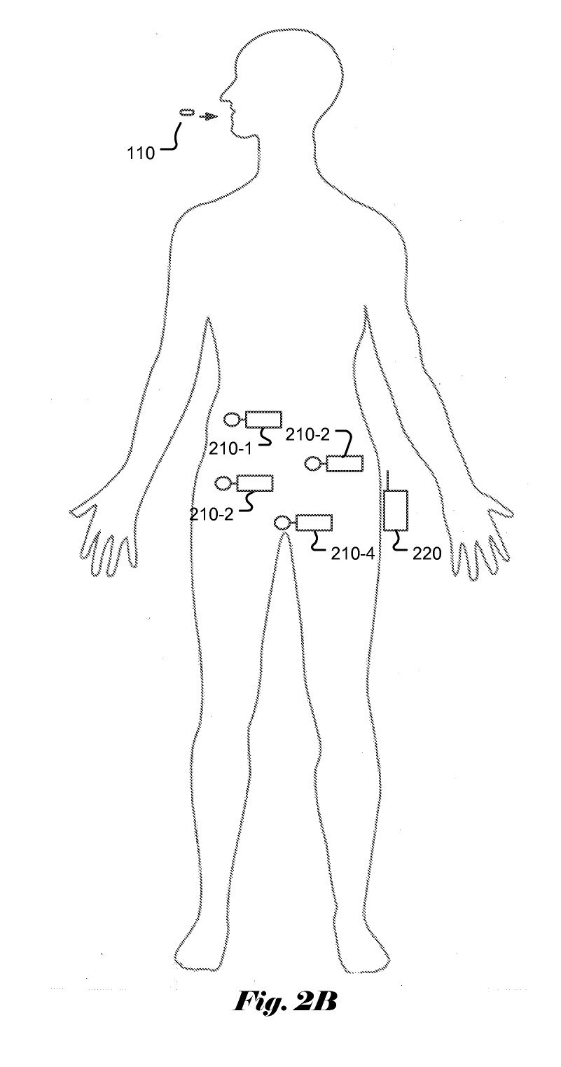

A medical capsule typically comprises a housing to enclose some electronic or mechanical parts required to provide intended operations. For example, a capsule camera may include a lens, an LED, and electronics for image capturing. Some parts inside the capsule will exhibit conductive or capacitive characteristics that are distinctive from the surrounding tissue of the living body. A sensing device may be used outside the living body to detect the proximity of the capsule. FIG. 2A illustrates the operational principle and the structure of a sensing device for detecting the proximity of a capsule inside the body. A sensing device 210 is placed outside the body where the sensing device comprises a radiating element 252 (also called transmitter head), a sensing element 254 (also called receiver head), and a control unit 230. The control unit 230 provides driving signal to the radiating element 252 through line 232. The radiating element 252 is made of conductive materials and forms a loop to allow electric current associated with the driving signal to flow through. The electric current flowing through the radiating element 252 creates an electromagnetic field 256. The electromagnetic field can penetrate the living body and causes an induced current 264, called Eddie current, on a conductive object 262, i.e. conductive parts inside the capsule. The induced Eddie current 264 will generate a secondary electromagnetic field 266 that may be sensed by the sensing element 254. Consequently, the existence of a conductive or capacitive object near the proximity of the sensing device will cause certain variations in the received field which can be used to evaluate the proximity data. The sensing element 254 is also made of conductive material. In order to increase the efficiency of the radiating element 252 and the sensing element 254, both elements may be formed of multiple loops. While a loop or a multi-loop coil is used as the radiating element and the sensing element, other shapes may also be used to radiate and receive signals. Beside the electromagnetic field is used in this configuration to induce a field, the electric field may also be used for the sensing purpose. For example, a dipole antenna may be used to radiate and to receive electric field to evaluate any surround field change caused by a capsule inside the living body.

The configuration of the radiating element 252 and the sensing element 254 as shown in FIG. 2A illustrates a case that both elements are substantially co-located. Therefore, the primary electromagnetic field 256 is directly coupled to the sensing element 254. As a result, the signals sensed by the sensing element 254 are a combination of the primary electromagnetic field 256 and the secondary electromagnetic field 266. It is also possible to use a configuration where the radiating element 252 and the sensing element 254 are separated apart and the coupling of the primary electromagnetic field from the radiating element to the sensing element may be greatly reduced. Shielding may also be incorporated into the transmitter head and receiver head design to reduce the coupling of the primary electromagnetic field from the transmitter head to the receiver head. As a result, the receiver head will be shielded from the primary field while the receiver head is still responsive to detect the induced field caused by an object near the receiver head.

FIG. 2B illustrates an example of capsule location determining system using multiple sensing devices 210-1 through 210-4 and a base station 220 to detect the location of a medical capsule 110 adapted to be ingested into the living body. In this example, four sensing devices 210-1 through 210-4 are placed over the abdomen area with the intention to determine the capsule location when it reaches the colon. More or less sensing devices may be used depending on the required accuracy and reliability of the location determination. The placement of the sensing devices also depends on intended application. For example, if the system is intended to determine when the capsule will arrive at the small intestine from the stomach, the sensing devices may be scattered around the joint area between the small intestine and the stomach. If the system is intended to determine when the capsule is about to exit from the living body, the sensing devices may be placed around the rectum and anus area. The base station may be carried by the patient or configured as a wearable device.

While the capsule location determining system can rely on sensed signal to evaluate the capsule location, the images captured by a capsule camera may also help to identify the capsule location. For example, Hepatic flexure and Splenic flexure, where the colon makes 90° turns, may show up differently in the images captured and can also be used as landmarks to identify the corresponding location of captured images. Furthermore, a timestamp may be used to associate with the images so that individual images may derive their respective locations based on images with known locations using an interpolation technique. By combining the location information evaluated by the present location determining system, image feature-based location detection and the interpolation technique, improved location detection can be achieved.

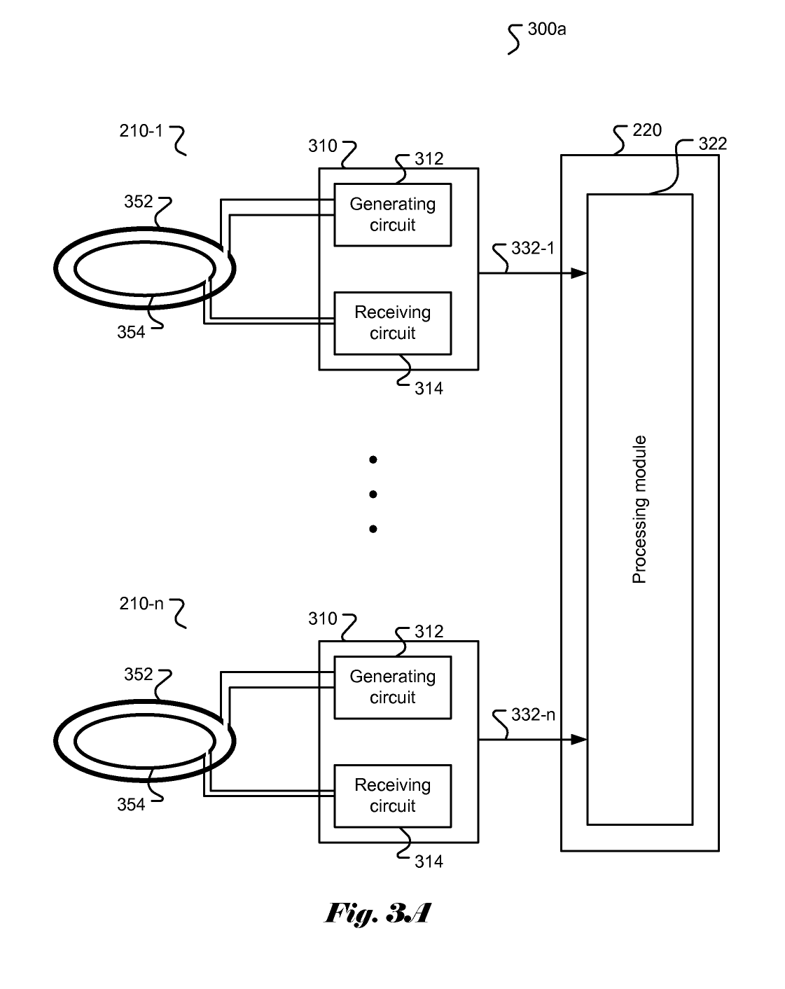

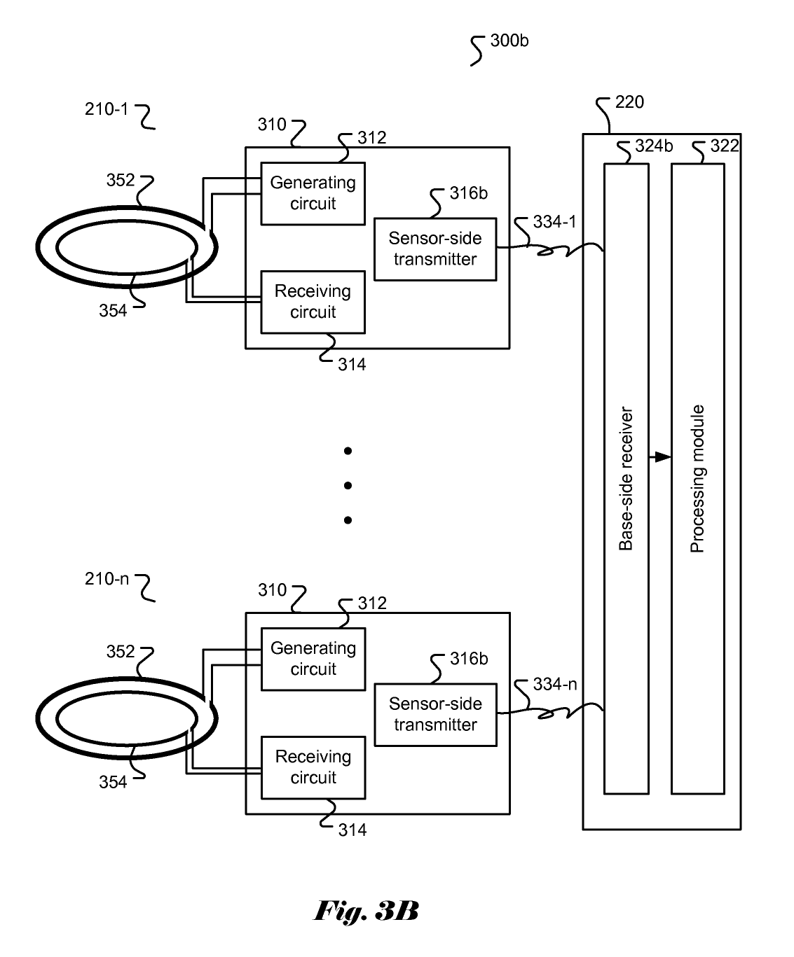

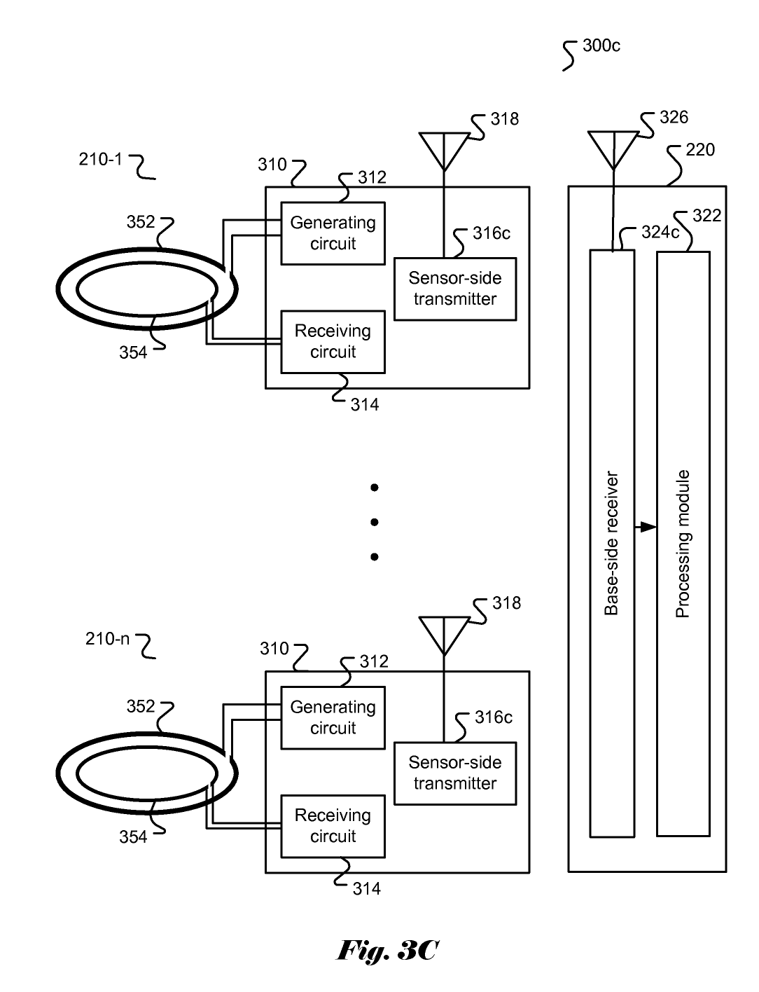

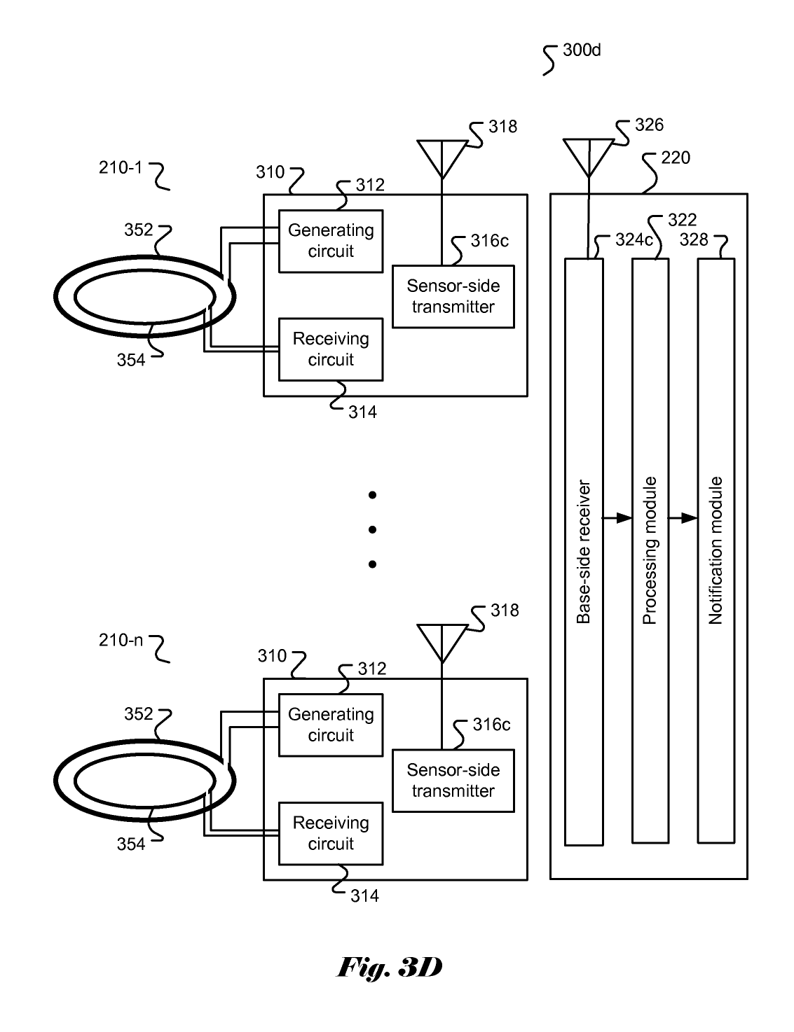

FIG. 3A illustrates an exemplary block diagram of location determining system 300a having a plurality of sensing devices 210-1 through 210-n and a base station 220. Each sensing device comprises a radiating element 352, a sensing element 354, a control unit 310, which comprises a generating circuit 312 and a receiving circuit 314. The generating circuit supplies a patterned signal to drive the radiating element. In the environment with multiple sensing devices, the fields radiated from the radiating elements of the sensing devices may cause interference among the sensing devices. A sensing element may receive inducing fields from radiating elements other than its respective radiating element. In order to avoid interference among the sensing devices, the driving signals provided to the radiating elements can be in a time-division multiplexing pattern. For example, the sensing devices can be operated sequentially according to a certain order such as looping from unit 210-1 to unit 210-n. Therefore, only one respective radiating element and sensing element will be active at a time. The patterned signal may be an alternating signal such as a sinusoidal or square wave at very low frequency (VLF). The use of very low frequency signal will allow the electromagnetic field to penetrate through the living body and to allow the sensing element to receive induced field. It is acknowledged that VLF in certain convention refers to frequency band from 3 kHz to 30 kHz. The frequency operable for the sensing device can be as low as a few kHz and as high as 300 kHz. However, it is preferred to use a non-audible frequency so as not to cause any annoying sound. Therefore, the VLF frequency used in this disclosure refers to frequency band from 15 kHz to 300 kHz. The induced field received by the sensing element will render itself as a phase shifted version of the driving signal. The quantity of phase shifted will provide indication of proximity of the object.

A pulse signal may also be used as the patterned signal. In this case, a short pulse signal is supplied to the radiating element to cause an inducing electromagnetic field. When a capsule exists in the proximity of the sensing device, the spread of the pulse caused by the induced field will change. The spread of the pulse is related to the length of delay as well as the speed of the decay. Usually, a train of pulses will be transmitted so that the measurement can be performed continuous to improve accuracy. While separate radiating element and sensing element and associated generating circuit and receiving circuit may be used, the elements and the circuits can be shared as well by switching quickly from a transmit mode to a receive mode. The spread of pulse can be measured in termed of time period from a reference time to a time that the amplitude of the received signal decays to a predetermined percentage of the maximum received signal. The reference time may be the instance that a pulse is generated by the generating circuit or is transmitted from the radiating element.

The driving signal can be a beat-frequency oscillator (BFO) type where the generating circuit provides a reference signal to the receiving circuit. The driving signal is an alternating signal having a frequency slightly different from that of the reference signal provided to the radiating element. For example, 100 kHz may be selected for the reference signal and 97.5 kHz may be selected for the driving signal. The 97.5 kHz is this example is the target frequency by design and the actual frequency is determined based on the inductance of the radiating element and the capacitance between the radiating element and the underlying area being detected. A conductive or capacitive object near the radiating element, also called the search head, will change the tuning characteristics of the radiating signal and causes a shift in the frequency of the radiating signal. The target beat frequency, i.e. the difference frequency, in this example is 2.5 kHz. When an object exists near the search head, the beat frequency may be shifted upward or downward. For example, the beat frequency may be lowered to 2.0 kHz that the bare human ear may be able to distinguish this lowered frequency from the target 2.5 kHz. It is noticed that there is no separate sensing element needed for the BFO system. Conceptually it may be regarded that the radiating element and the sensing element are shared and the generating circuit and the receiving circuit are also shared according to the sensing device convention of FIG. 2A. The amount of frequency shifted provides an indication of proximity of an object, i.e. the capsule in this case.

The signal receiving circuit 314 is coupled to the sensing element to receive the induced field and to provide a recovered signal. The receiving circuit 314 may also include circuits necessary to provide a reliably recovered signal, such as timing circuit, noise reduction circuit and signal enhancement circuit. For the VLF system, the receiving circuit has to convert the received induced field into a voltage signal corresponding to the induced field. The recovered signal is subject to further processing to provide the needed proximity information. The further processing may be performed by the receiving circuit of the sensing device or the processor of the base station. The further processing, for example, may be comparing the recovered signal with the patterned signal to derive the phase difference which is an indication of proximity data of the capsule for the VLF system. The receiving circuit may be configured to perform this further processing. When the PI system is used, the receiving circuit may measure the time period between the moment a pulse is transmitted and the moment that the amplitude of the received signal decays to a pre-determined level. When the BFO system is used, the receiving circuit may receive the induced field and mix it with the reference signal to produce the audible beat frequency signal as an indication of proximity. The receiving circuit may further extract the beat frequency which can be directly used by a digital processor and any other digital means to assess the proximity instead of being listened by a human being.

The signal lines 332-1 through 332-n indicate that the base station 220 collects information from the sensing devices to determine the location of the medical capsule using a processing module 322. The processing module can be implemented using a computer, a microcontroller, a digital signal processing unit, a field programmable gate array (FPGA), an application specific integrated circuit, or a combination of these. Furthermore, program codes, micro-codes, machine codes, or other machine-executable codes may be used to control the respective hardware to perform the required processing. The collected information may be associated with the recovered signal. In the cases of PI and BFO, the proximity information can be derived based on the recovered signal alone without referring to the patterned signal. The collected information may also be associated with both the patterned signal and the recovered signal such as the case of VLF. The base station can use a profile of the proximity data over time from the sensing devices as a whole to determine the location of the medical capsule. For example, the sensing devices 210-1 and 210-2 of FIG. 2B are placed at locations corresponding to both ends of the ascending colon. When the unit 210-2 picks up a strongest proximity signal, it indicates that the medical capsule is near the lower end of the ascending colon. After some time, both units 210-1 and 210-2 may pick up proximity signal of equal strength; it indicates that the medical capsule travels to the middle of the ascending colon. The base station may use interpolation technique to estimate the location of the medical capsule between known locations.

FIG. 3B illustrates a capsule location determining system 300b where the sensing devices are connected to the base station through wired links 334-1 through 334-n. A sensor-side transmitter 316b at each sensing device is used to transmit the collected information to the base station where the base station uses a base-side receiver 324b to receive the information. The transmitter and receiver for the wired link can be very simple and low cost. For example, a deriver circuit can be used as the transmitter and a buffer can be used as the receiver. An implication of the wired links is that physical wires are needed between the base station and all the sensing devices. When the number of sensing devices is large, the wiring can be cumbersome. FIG. 3C illustrates an alternative capsule location determining system 300c where wireless link is used to connect between the base station and the sensing devices. Each sensing device uses a wireless transmitter 316c to transmit collected information to the base station where the base station uses a wireless receiver 324c to receive the collected information. The wireless connectivity will provide great convenience for practicing the present invention. However, it is anticipated that it will incur a higher system cost and caution has to be exercised so that the wireless link will not cause any interference to any other medical equipment and the other way around. FIG. 3D illustrates yet another embodiment 300d according to the present invention. The base station in FIG. 3D includes a notification module 328 which may provide a notification signal based on the location information. For example, when the medical capsule is near the rectum or anus, it may be desirable to send a notification signal to alert the event that the medical capsule is about to exit from the human body. In another example, the base station may determine that the medical capsule is stuck at one location for too long and sends a notification signal to alert a medical personnel. The notification module may generate an audible signal and/or a visually alerting signal, such as a flashing bright light or red light. Alternatively, the notification module may send the notification signal wirelessly to a remote location. For example, the notification module may be connected to a Wi-Fi network and the notification signal may be sent to an attending medical personnel. In yet another alternative arrangement, the notification module may be coupled to a cellphone network and the notification signal may be delivered as a computer-based or pre-recorded voice message to an attending personnel. While FIG. 3D illustrates that the notification module is used in a wireless system, the notification module may also used in a wired system.

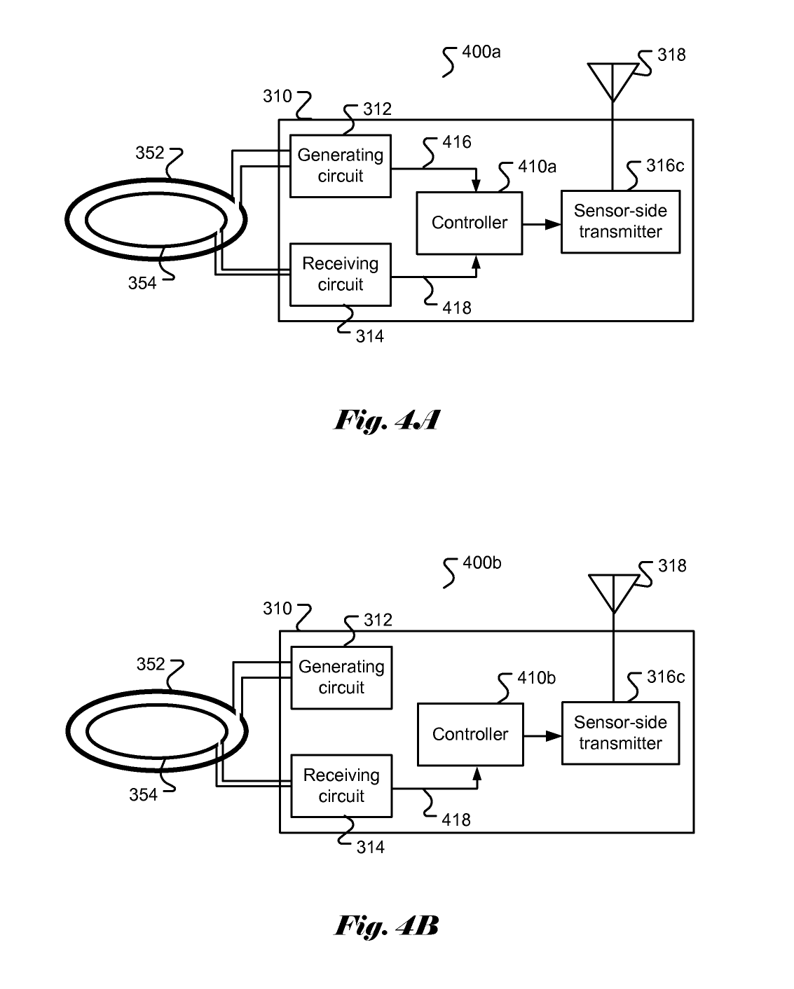

FIG. 4A illustrates a sensing device 400a having a controller 410a where the controller is coupled to both the patterned signal via signal line 416 and the recovered signal via signal line 418. As described previously, the base station estimates capsule location based on collected information from the sensing devices. The recovered signal at the sensing device may take up noticeable bandwidth to transmit to the base station. This may be of concern when wireless links are used between the base station and the sensing devices. For example, each recovered signal for the VLF case may require a bandwidth in the order of several hundred kilo-Hertz when the signal is transmitted using direct amplitude modulation. Furthermore, the patterned signal has to be delivered to the base station as well in order to derive the phase difference between the two signals for the VLF system. However, the phase difference may be evaluated using the controller at each sensing device and the phase difference represents a very small amount of digital information to be transmitted to the base station. Consequently, the use of a controller can greatly relieve the communication bandwidth issue, particularly for the wireless links between the base station and the sensing devices. FIG. 4B illustrates an alternative sensing device 400b having a controller 410b where the controller is coupled to the recovered signal through signal line 418. The controller may measure the spread of pulse for the PI system and the beat frequency for the BFO system. Therefore, only the extracted pulse spread and the beat frequency are needed to be transmitted instead of the recovered signal. Again, the use of the controller can greatly relieve the communication bandwidth issue, particularly for the wireless links between the base station and the sensing devices.

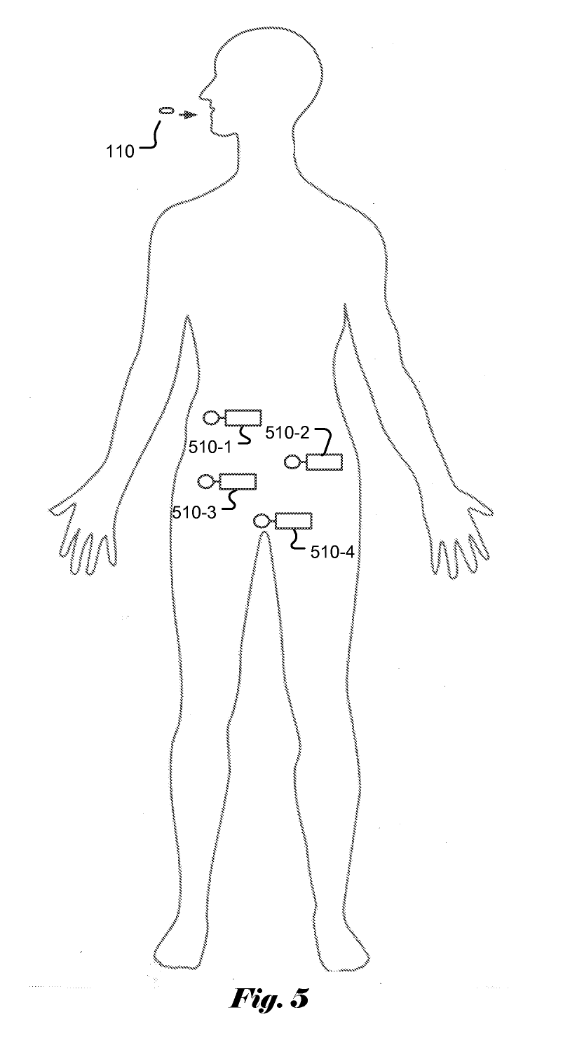

FIG. 5 illustrates an alternative capsule location system using multiple sensing devices 510-1 through 510-4 to detect the location of a medical capsule 110 adapted to be ingested into the living body. The system in FIG. 5 illustrates a system without a base station to make a centralized decision. The sensing devices may function in a distributed fashion to process information jointly. The system is considered as an autonomous system because there is no centralized unit. Each sensing device will transmit collected information associated with the device to other devices. Also, each device will receive collected information from other devices. Therefore, each device needs a transceiver to transmit information to other devices and receive information from other devices. Since each device receive the same information as the base station in FIG. 2B, each sensing device can make the same decision as a base station would make if each sensing device has sufficient processing capability. The advantage of the distributed system is that there is no need for a base station. However, each sensing device will require increased processing power. Nevertheless, in light of advancement in very large scale integrated (VLSI) circuit technology, the requirement to accommodate the increased processing power will only have a small impact on the system cost and power consumption.

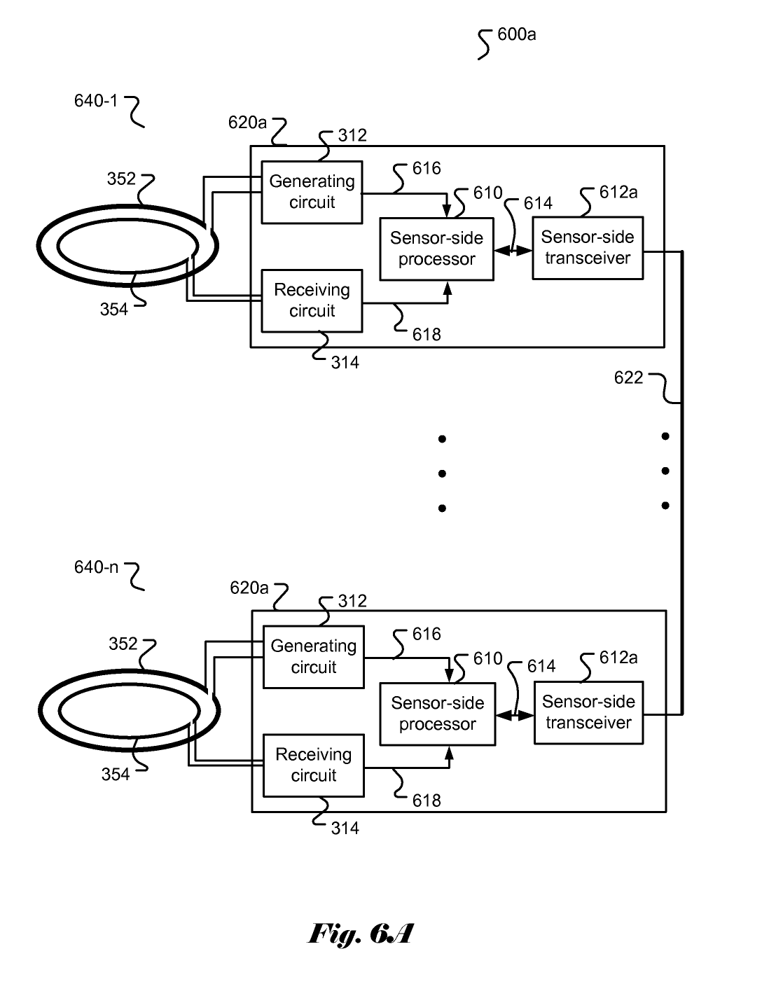

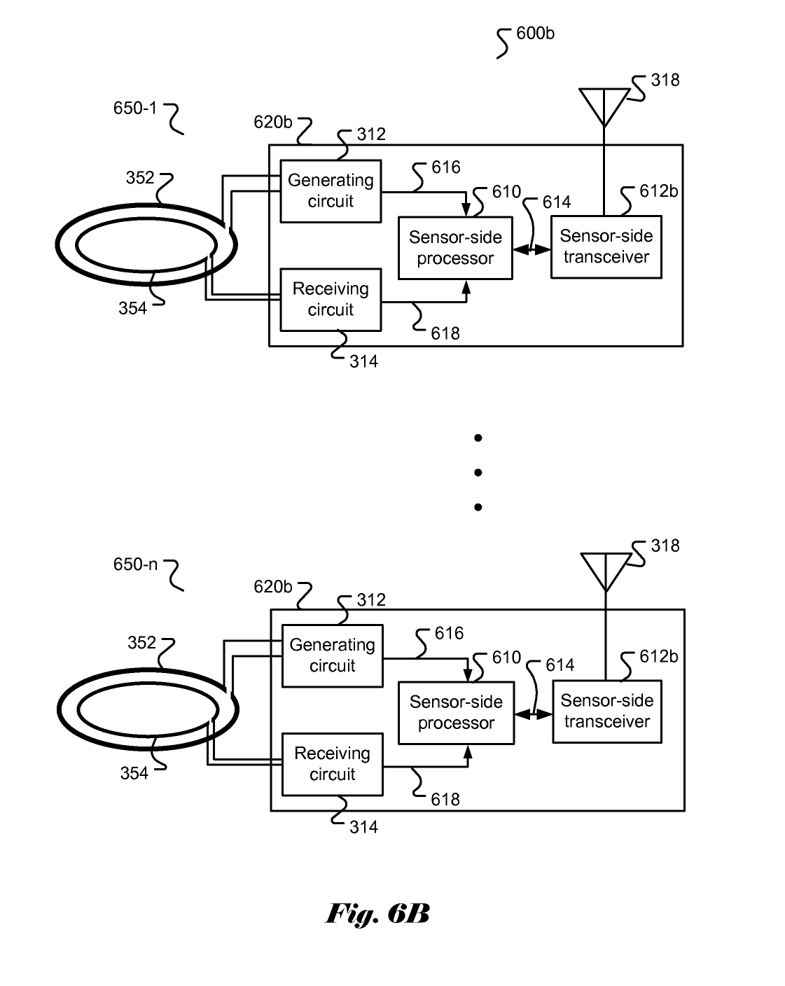

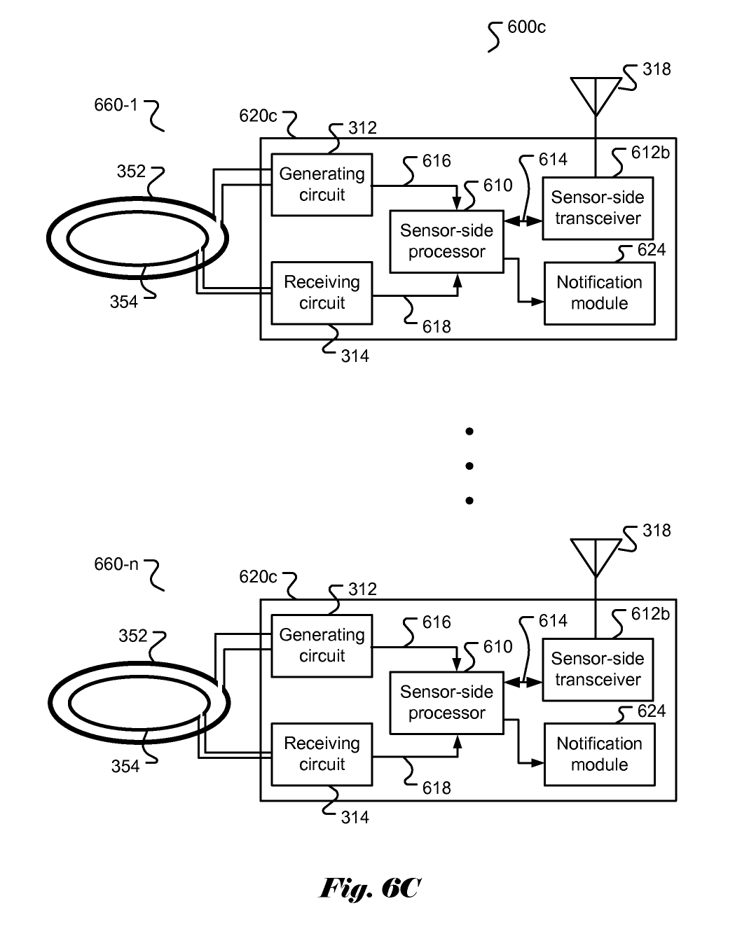

FIG. 6A illustrates an example of distributed capsule location determining system 600a where the sensing devices 640-1 through 640-n are connected through a wired link. Each sensing device comprises a radiating element 352, a sensing element 354, and a control unit 620a. The control unit 620a comprises a generating circuit 312, a receiving circuit 314, a sensor-side processor 610 and a sensor-side transceiver 612a. A wired link 622 is used to connect all sensing devices. The sensor-side processor 610 is coupled to both the patterned signal via signal line 616 and the recovered signal via signal line 618. Each sensing device evaluates the proximity information using the sensor-side processor and transmits the proximity information to other devices using the transceiver 612a. Each sensing device also receives information from other sensing devices using the transceiver 612a. The received information is passed to the processor 610 through interface 614. Each sensing device has proximity information from all sensing devices and each sensing device can estimate capsule location based on the proximity information from all sensing devices. While each processor evaluates proximity information and transmit it to other sensing devices, each sensing device may also transmit recovered information to other sensing devices. Nevertheless, it would be more computationally effective that each sensing device evaluates the proximity information and transmit it to other sensing devices. The wired transceiver 612a can be simply a combination of driver circuit and buffer. In order to accommodate the communication among multiple sensing devices, a certain communication protocol may be required. Communication protocol for shared access among multiple devices is well known in the art.

FIG. 6B illustrates an alternative distributed capsule location determining system using multiple sensing devices 650-1 through 650-n. The control unit 620b is substantially the same as the control unit 620a of FIG. 6A except that a wireless transceiver 612b is used. The wireless transceivers form a wireless network among the sensing devices. FIG. 6C illustrates another distributed capsule location determining system using multiple sensing devices 660-1 through 660-n, where each sensing device further comprises a notification module 624. The notification module 624 provides a notification signal based on capsule location. Alternatively, a notification module can be included in only one designated sensing device instead of all sensing devices to save cost.

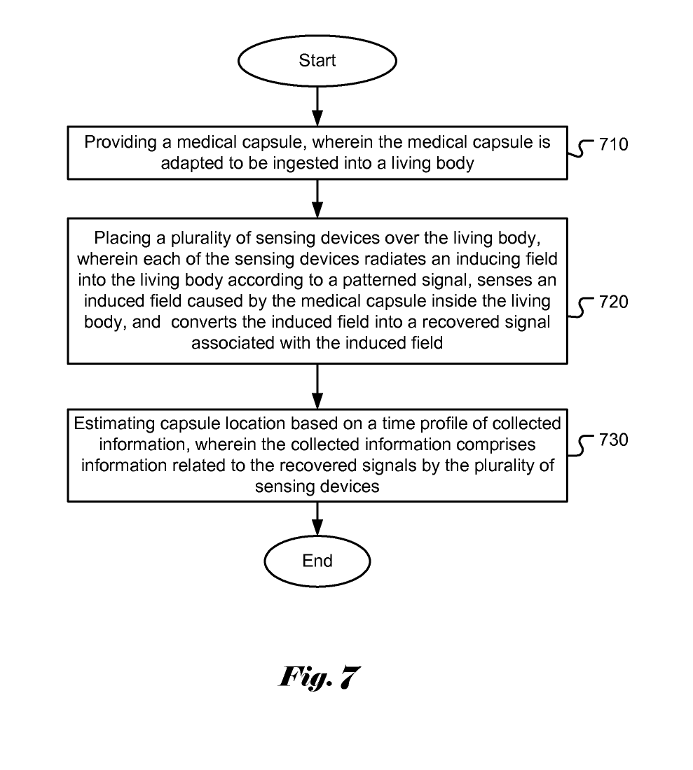

FIG. 7 illustrates a flowchart corresponding to a method of estimating capsule location using a plurality of sensing devices and a base station. The method for locating a medical capsule inside a living body comprising: providing a medical capsule, wherein the medical capsule is adapted to be ingested into a living body as shown in step 710, and placing a plurality of sensing devices over the living body as shown in step 720. Each of the sensing devices radiates an inducing field into the living body according to a patterned signal, senses an induced field caused by the medical capsule inside the living body, and converts the induced field into a recovered signal associated with the induced field. The method further comprises estimating capsule location based on a time profile of collected information as shown in step 730. The collected information comprises information related to the recovered signals by the plurality of sensing devices.

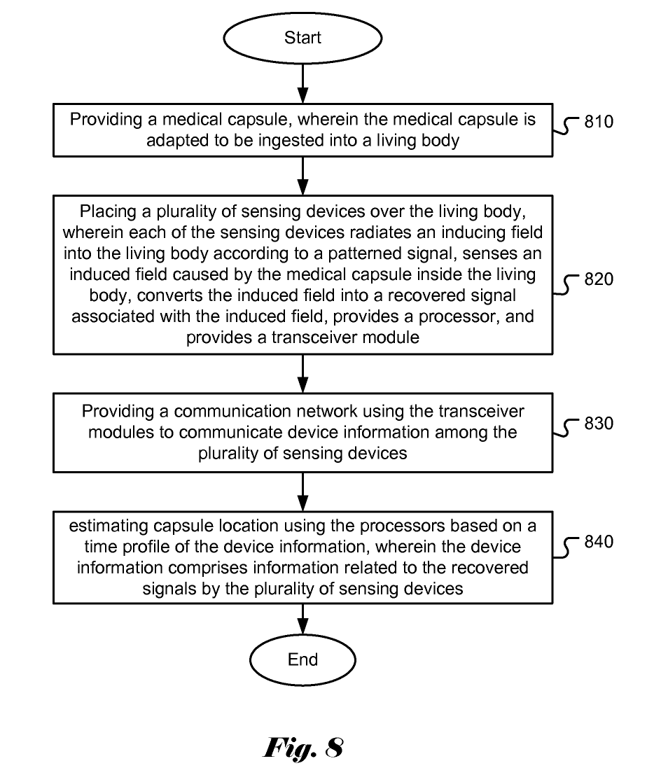

FIG. 8 illustrates a flowchart corresponding to an alternative method of estimating capsule location using a plurality of sensing devices. The method for locating a medical capsule inside a living body comprises: providing a medical capsule, wherein the medical capsule is adapted to be ingested into a living body as shown in step 810, and placing a plurality of sensing devices over the living body as shown in step 820. Each of the sensing devices radiates an inducing field into the living body according to a patterned signal, senses an induced field caused by the medical capsule inside the living body, converts the induced field into a recovered signal associated with the induced field, provides a processor, and provides a transceiver module. The method further comprises: providing a communication network using the transceiver modules to communicate device information among the plurality of sensing devices as shown in step 830, and estimating capsule location using the processors based on a time profile of the device information, wherein the device information comprises information related to the recovered signals by the plurality of sensing devices as shown in step 840.

The above detailed description illustrates the specific embodiments of the present invention and is not intended to be limiting. Numerous modifications and variations within the scope of the invention are possible. The present invention is set forth in the following claims.

1. A location determining system for a medical capsule, comprising:
a medical capsule adapted to be ingested into a living body;
a plurality of sensing devices; and
a base station;
wherein each of the sensing devices comprises (i) a radiating element to radiate an inducing field into the living body, (ii) a signal generating circuit to provide a patterned signal to drive the radiating element, (iii) a sensing element to sense an induced field caused by the medical capsule inside the living body, and (iv) a signal receiving circuit coupled to the sensing element to receive the induced field and to provide a recovered signal associated with the induced field; and
wherein the base station comprises a processing module to estimate capsule location based on a time profile of collected information, wherein the collected information comprises information related to the recovered signals by the plurality of sensing devices.

2. The location determining system of claim 1, wherein the collected information further comprises information related to the patterned signal.

3. The location determining system of claim 1, wherein each of the plurality of sensing devices further comprises a controller to evaluate proximity data to include in the collected information.

4. The location determining system of claim 1, wherein the base station further comprises a notification module to provide notification information based on the capsule location.

5. The location determining system of claim 1, wherein each of the plurality of sensing devices further comprises a sensor-side transmitter and the base station further comprises a base-side receiver, wherein the sensor-side transmitter and the base-side receiver communicate through a wired link.

6. The location determining system of claim 1, wherein each of the plurality of sensing devices further comprises a sensor-side transmitter and the base station further comprises a base-side receiver, wherein the sensor-side transmitter and the base-side receiver communicate through a wireless link.

7. The location determining system of claim 5, further comprising:
a base-side transmitter in the base station; and
a sensor-side receiver in each sensing device;
wherein the base-side transmitter and the sensor-side receivers communicate through a second wired link to provide system management for the plurality of sensing devices by the base station.

8. The location determining system of claim 6, further comprising:
a base-side transmitter in the base station; and
a sensor-side receiver in each sensing device;
wherein the base-side transmitter and the sensor-side receivers communicate through a second wireless link to provide system management for the plurality of sensing devices by the base station.

9. The location determining system of claim 1, wherein the base station provides capsule management information to the medical capsule.

10. The location determining system of claim 9, wherein the capsule management information comprises a timestamp.

11. The location determining system of claim 9, wherein the capsule management information comprises a command.

12. The location determining system of claim 9, wherein the capsule management information comprises capsule location.

13. The location determining system of claim 1, wherein the patterned signal is a very low frequency (VLF) signal.

14. The location determining system of claim 1, wherein the patterned signal is a pulse induction (PI) signal.

15. The location determining system of claim 1, wherein the patterned signal is a beat-frequency oscillator (BFO) signal.

16. The location determining system of claim 3, wherein the patterned signal is a very low frequency (VLF) signal and the proximity data is associated with phase relationship between the patterned signal and the recovered signal.

17. The location determining system of claim 3, wherein the patterned signal is a pulse induction (PI) signal and the proximity data is associated with pulse spread of the recovered signal.

18. The location determining system of claim 3, wherein the patterned signal is a beat-frequency oscillator (BFO) signal and the proximity data is associated with frequency difference between a reference signal and the recovered signal.

19. The location determining system of claim 1, wherein the radiating element and the sensing element of the sensing device are substantially co-located to form a combined sensing head.

20. The location determining system of claim 1, wherein the inducing field is an electromagnetic field.

21. A method for locating a medical capsule inside a living body, the method comprising:
providing a medical capsule, wherein the medical capsule is adapted to be ingested into a living body;
placing a plurality of sensing devices over the living body, wherein each of the sensing devices (i) radiates an inducing field into the living body according to a patterned signal, (ii) senses an induced field caused by the medical capsule inside the living body, and (iii) converts the induced field into a recovered signal associated with the induced field; and
estimating capsule location based on a time profile of collected information, wherein the collected information comprises information related to the recovered signals by the plurality of sensing devices.

22. The method of claim [0014], wherein the estimating capsule location is performed using a processor in a base station.

23. The method of claim [0014], further comprising:
providing a notification signal based on the capsule location.

24. The method of claim [0014], wherein the collected information further comprises information related to the patterned signal.

25. The method of claim [0014], further comprising: providing capsule management information from the base station to the medical capsule.

26. The method of claim 25, wherein the capsule management information comprises a timestamp.

27. The method of claim 25, wherein the capsule management information comprises a command.

28. The method of claim 25, wherein the capsule management information comprises capsule location.

29. The method of claim [0014], wherein the plurality of sensing devices are operated in a time-division multiplexing pattern.

30. A location determining system, comprising:
a medical capsule adapted to be ingested into a living body;
a plurality of sensing devices, wherein each sensing device comprises:
(i) a radiating element to radiate an inducing field into the living body;
(ii) a signal generating circuit to provide a patterned signal to drive the radiating element;
(iii) a sensing element to sense an induced field caused by the medical capsule inside the living body;
(iv) a signal receiving circuit coupled to the sensing element to receive the induced field and to provide a recovered signal associated with the induced field;
(v) a processor; and
(vi) a transceiver module;
wherein a communication network is formed using the transceiver modules to communicate device information among the plurality of sensing devices; and
wherein the processors are used to estimate capsule location based on a time profile of the device information related to the recovered signals by the plurality of sensing devices.

31. The location determining system of claim 30, wherein the device information further comprises information related to the patterned signal.

32. The location determining system of claim 30, wherein each of the plurality of sensing devices comprises a notification module to provide a notification signal according to the estimated capsule location.

33. A method for locating a medical capsule inside a living body, the method comprising:
providing a medical capsule, wherein the medical capsule is adapted to be ingested into a living body;
placing a plurality of sensing devices over the living body, wherein each of the sensing devices (i) radiates an inducing field into the living body according to a patterned signal, (ii) senses an induced field caused by the medical capsule inside the living body, (iii) converts the induced field into a recovered signal associated with the induced field, (iv) provides a processor, and (v) provides a transceiver module;
providing a communication network using the transceiver modules to communicate device information among the plurality of sensing devices; and
estimating capsule location using the processors based on a time profile of the device information, wherein the device information comprises information related to the recovered signals by the plurality of sensing devices.
